(19) United States
(12) Patent Application Publication
(10) Pub. No.: US 2016/0298086 A1
(43) Pub. Date: Oct. 13, 2016

(54) METHOD FOR DETERMINING THE CARDIO-GENERATIVE POTENTIAL OF MAMMALIAN CELLS

(71) Applicants: Mayo Foundation for Medical Education and Research, Rochester, MN (US); Cardio3 Biosciences SA, Mont-Saint-Giubert (BR)

(72) Inventors: Andre Terzic, Rochester, MN (US); Atta Behfar, Rochester, MN (US); Roland Gordon-Beresford, La Hulpe (BE); Vinciane Gaussin, Wezembeek-Oppem (BE)

(21) Appl. No.: 15/188,582

(22) Filed: Jun. 21, 2016

Related U.S. Application Data
(63) Continuation of application No. 13/321,100, filed on Jan. 6, 2012.
Application No. 13/321,100 is a continuation of application No. PCT/US2009/044751, filed on May 20, 2009.

(51) Int. Cl. C12N 5/077; A61K 35/34; C12Q 1/68

(52) U.S. Cl. CPC C12N 5/0657; C12Q 1/6876; A61K 35/34; C12N 2506/1353; C12N 2501/15; C12N 2501/155; C12N 2501/115; C12N 2501/105; C12N 2501/16; C12N 2501/23; C12N 2501/734; C12N 2501/999; C12Q 2600/158; C12Q 2600/136

(57) ABSTRACT

This document is related to a method for determining the cardio-generative potential of mammalian cells which comprises the assessment of a CARdiac generation Potential Index (CARPI) as a function of the quantification of the expression of genes of said cells. It also relates to a method for quantitatively assessing the modification of this cardio-generative potential and the cardiogenic potential of a treatment aiming at cellular differentiation.

### CROSS-REFERENCE TO RELATED APPLICATIONS

This application is a continuation of U.S. application Ser. No. 13/321,100, filed Jan. 6, 2012, which is a National Stage application under 35 U.S.C. §371 and claims benefit of International Application No. PCT/US2010/035616, filed May 20, 2010, which claims the benefit of priority to International Application Serial No. PCT/US2009/044751, filed May 20, 2009. The disclosure of the prior applications are considered part of (and are incorporated by reference in) the disclosure of this application.

### FIELD OF THE INVENTION

The present invention relates to the treatment of heart disease disorders through injection of mammalian cells. In particular, it relates to a method for quantitatively assessing the cardio-generative potential of mammalian cells, thereby allowing a good predictability of the success of repairing a heart in need. It also relates to a method for quantitatively assessing the modification of this cardio-generative potential and the cardiogenic potential of a treatment aiming at cellular differentiation, and a computer device comprising a processor, and a memory encoding one or more non-neural network programs coupled to the processor, wherein said programs cause the processor to perform a method, said method comprising calculating a CARPI.

### STATE OF THE ART

Cardiovascular diseases are leading cause of morbidity and mortality worldwide, despite advances in patient management. In contrast to tissues with high reparative capacity, heart tissue is vulnerable to irreparable damages. Cell-based regenerative cardiovascular medicine is now being pursued in the clinical setting to address heart disease disorders.

Recent advent of stem cell biology extends the scope of current models of practice from traditional palliation towards curative repair. Typically, clinical experience has been based on adult stem cells delivered in an unaltered state. First generation biologics are naive human stem cells, identified as readily accessible cytotypes. It has been shown that a few individuals improve on delivery of naive human stem cells. The state of the art in the field of naive cell transplantation in the heart of humans was described inter alia in the review carried by Abdel-Latif A. et al. ‘Adult bone marrow-derived cells for cardiac repair: a systematic review and meta-analysis.’ Arch Intern Med. (2007) 167:989-997, and citations therein.

To improve clinical outcome, second-generation stem cell therapies were developed to guide naive human stem cells towards the cardiac lineage prior to injection into the patient. In the review by Behfar et al. ‘Guided stem cell cardiopoietic: Discovery and translation’ J. Mol. and Cell. Cardiology (2008) 45: 523-529, the concept of using cardiac precursor cells, such as cardiopoietic cells, for heart regeneration was discussed.

Cardiopoietic cells have a unique phenotype: they are characterized by nuclear translocation of Nkx2.5 and MEF2C polypeptides, combined to the absence of detectable sarcomeric proteins. This cardiopoietic status corresponds to an intermediate cell phenotype, i.e. committed to the cardiac lineage but not yet fully differentiated. Non-detectable level of sarcomeric protein expression is a unique feature of cardiopoietic cells which distinguishes them from contractile and sarcomeric-containing cardiomyocyte-like cells derived from stem cells and described in other applications such as by Chunhui Xu (U.S. 2005/0164382) and Lough et al (U.S. 2002/0061837).

Increased protein content of a transcription factor may not imply its subcellular localization, which could be either cytoplasmic or nuclear. Nuclear translocation of Nkx2.5 and MEF2C polypeptides is necessary for definitive cardiac lineage commitment. This is further explained in Behfar A. et al, (Derivation of a cardiopoietic population from human mesenchymal stem cells yields cardiac progeny, Nature Clinical Practice, 2006, 3:S78-S82). Although nuclear translocation may be qualitatively observed by immunocytochemistry or immunohistochemistry, techniques such as western blotting or Fluorescence Activated Cell Sorting (FACS) that look at total protein content are not suitable for quantitative assessment of the subcellular distribution of a polypeptide. The observation of subcellular distribution of a polypeptide, as described in U.S. 2008/0019944, is not only qualitative but also time-consuming in the industrial perspective and operator-dependent. Thus clinical outcome, i.e. the cardio-generative potential of these “first-generation” naive stem cells and “second-generation” guided stem cells could not be readily predicted prior to injection.

A method to quantitatively assess the cardio-generative potential of mammalian cells remained to be proposed.

The present invention now provides such a predictive method for determining the cardio generative potential of mammalian cells which comprises the quantitative assessment of a CARdiac generation Potential Index (CARPI) as a function of the quantification of the expression of genes of said cells. It also addresses the quantitative assessment of the modification of the cardio generative potential of mammalian cells and the cardiogenic potential of a treatment aiming at cellular differentiation.

Definitions

Within the frame of the present document, and unless indicated to the contrary, the terms designated below between quotes have the following definitions.

The ‘cardio-generative potential’ of a cell designates the ability of this cell to succeed to generate heart cells, for instance cardiac myocytes.

‘Cardiopoietic cells’ are an intermediate cell phenotype, i.e. committed to the cardiac lineage but not yet fully differentiated. Cardiopoietic cells are characterized by nuclear translocation of Nkx2.5 and MEF2C, combined to the absence of detectable sarcomeric proteins (Behfar et al. ‘Derivation of a cardiopoietic population from human mesenchymal stem yields progeny’, Nature Clin. Pract., Cardiovasc. Med. (2006) 3: S78-S82). Cardiopoietic cells retain a proliferative capacity. Cardiopoietic cells can be derived from stems cells including for example, human adult mesenchymal stem cells (Terzic et al. U.S. 2008/0019944), mouse embryonic stem cells (Behfar et al, ‘Cardiopoietic programming of embryonic stem cells for tumour-free heart repair’ J Exp Med 2007 204: 405-420), embryonic-like stem cells, inducible pluripotent stem cells, umbilical cord blood cells, resident cardiac stem cells and the like, or any other adapted source (provided their production implies no human embryo destruction).

A ‘cocktail’ or ‘cardiogenic cocktail’ designates a composition containing at least two cardiogenic substances.

A ‘cardiogenic treatment’ is a treatment which improves the cardio-generative potential of a cell. Example of such treatment consists in putting said cell in contact with a cocktail. Examples of such cocktails comprise at least two substances selected in the group consisting of growth factors, cytokines, hormones and combinations thereof. Said at least two substances may be selected in the group consisting of bone morphogenetic proteins (BMP) such as BMP-1, BMP-2, BMP-5, BMP-6; epidermal growth factor (EGF); erythropoietin (EPO); fibroblast growth factors (FGF) such as FGF-1, FGF-4, FGF-5, FGF-12, FGF-13, FGF-15, FGF-20; granulocyte-colony stimulating factor (G-CSF); granulocyte-macrophage colony stimulating factor (GM-CSF); growth differentiation factor-9 (GDF-9); hepatocyte growth factor (HGF); insuline-like growth factor (IGF) such as IGF-2; myostatin (GDF-8); neurotrophins such as NT-3, NT-4, NT-1 and nerve growth factor (NGF); platelet-derived growth factor (PDGF) such as PDGF-beta, PDGF-AA, PDGF-BB; thrombopoietin (TPO); transforming growth factor alpha (TGF-α); transforming growth factors β (TGF-β) such as TGF-β1, TGF-β2, TGF-β3; vascular endothelial growth factor (VEGF) such as VEGF-A, VEGF-C; TNF-α; leukemia inhibitory factor (LIF); interleukin 6 (IL-6); retinoic acid; stromal cell-derived factor-1 (C SDF-1); brain-derived neurotrophic factor (BDNF); periostin; angiotensin II; Flt3 ligand; glial-derived neurotrophic factor; heparin; insulin-like growth factor binding protein-3; insulin-like growth factor binding protein-5; interleukin-3; interleukin-8; midkine; progesterone; putrescine; stem cell factor; Wnt1; Wnt3a; Wnt5a; caspase-4; chemokine ligand 1; chemokine ligand 2; chemokine ligand 5; chemokine ligand 7; chemokine ligand 11; chemokine ligand 20; haptoglobin; lectin; cholesterol 25-hydroxylase; syntaxin-8; syntaxin-11; ceruloplasmin; complement component 1; complement component 3; integrin alpha 6; lysosomal acid lipase 1; β-2 microglobulin; ubiquitin; macrophage migration inhibitory factor; cofilin; cyclophillin A; FKBP12; NDPK; profilin 1; cystatin C; calcyclin; stanniocalcin-1; PGE-2; mpCCL2; IDO; iNOS; HLA-G5; M-CSF; angiopoietin; PIGF; MCP-1; extracellular matrix molecules; CCL2 (MCP-1); CCL3 (MIP-1α); CCL4 (MIP-1β); CCL5 (RANTES); CCL7 (MCP-3); CCL20 (MIP-3α); CCL26 (eotaxin-3); CX3CL1 (fractalkine); CXCL5 (ENA-78); CXCL11 (i-TAC); CXCL1 (GROα) CXCL2 (GROβ); CXCL8 (IL-8); CCL10 (IP-10); and combinations thereof.

A ‘cocktail-guided cell’ or a ‘cell guided towards cardiac differentiation’ is a cell which has been put into contact with a cocktail.

‘Differentiation’ is the process by which a less specialized cell becomes a more specialized cell.

‘Ejection fraction’ means the fraction of blood pumped out during a heartbeat. Without a qualifier, the term ejection fraction refers specifically to that of the left ventricle (left ventricular ejection fraction or LVEF).

As used in the subject specification, the singular forms ‘a’, ‘an’ and ‘the’ include plural aspects unless the context clearly dictates otherwise. Thus, for example, reference to ‘a stem cell’ includes a single cell, as well as two or more cells; reference to ‘an agent’ or ‘a reagent’ includes a single agent or reagent, as well as two or more agents or reagents; reference to ‘the invention’ or ‘an invention’ includes single or multiple aspects of an invention; and so forth.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### SUMMARY OF THE INVENTION

The invention provides a method for determining the cardio-generative potential of mammalian cells or cardiogenic potential of a treatment which comprises the assessment of a CARdiac generation Potential Index (CARPI) as a function of the quantification of the expression of genes of said cells.

Preferably, the CARPI is a function of the quantification of messenger RNA (mRNA) levels of specific genes of said cells.

Preferably, at least one gene is chosen from the group consisting of Nkx2.5, Tbx5, MEF2C, GATA4, GATA6, Mesp1, FOG1, FOG2, Flk1, homologues thereof in mammals and combinations of these genes. The cells may be cardiac progenitor cells. They may also be somatic, germ, umbilical cord blood, cardiac progenitor, embryonic, and/or genetically modified cells.

In some cases, the cells can belong to one individual, and a CARPI can be assessed for those cells before and after exposing the cells to any cardiogenic treatment.

In another embodiment, a CARPI is assessed for cells of an individual or group of individuals versus another individual or group of individuals.

In a method particularly preferred, the CARPI is a multivariate equation where the expression of genes at the mRNA level is quantified as variables.

The equation is preferably chosen from the group consisting of polynomials functions, transcendental functions, and combinations thereof.

In a particular embodiment of a method provided herein a CARPI is measured to quantitatively assess the cardiogenic potential of a treatment.

According to one embodiment of a method provided herein, the CARPI may be put into correlation with a parameter of cardiac function.

The invention also relates to a computer device comprising a processor, and a memory encoding one or more programs coupled to the processor, wherein the one or more programs cause the processor to perform a method, said method comprising calculating a CARPI.

### BRIEF DESCRIPTION OF THE DRAWING

FIG. 1 shows in Y ordinate the CARPI, in arbitrary units (AU), calculated for both naive human MSC (hMSC) and cocktail guided-hMSC (CP-hMSC) on the basis of quantification of the expression of genes at the mRNA level and in X ordinate the change of LVEF (AEF) in percent prior and after injection in mouse infarcted hearts. Black symbols represent individual data; open symbols represent averaged data (Avg).

### DETAILED DESCRIPTION OF THE INVENTION

### EXAMPLE 1

Bone marrow samples were harvested from patients undergoing coronary artery bypass for ischemic heart disease. Patients provided informed consent, as approved by competent Institutional Ethics Committees.

Mesenchymal stem cells were recruited by plating of raw bone marrow on plastic dishes, with a wash at 12 h, selecting adhesive cells with identity confirmed by Fluorescence-Activated Cell Sorting (FACS) analysis using the CD34⁻/CD45⁻/CD133⁺ marker panel. Cells were further cultured and expanded at 37° C. in DMEM supplemented with 5% human platelet lysate (Mayo Clinic Blood Bank, Rochester, Minn.).

Naive human bone marrow-derived mesenchymal stem cells were cultured in either platelet lysate or serum supplemented with a cardiogenic cocktail consisting in TGFβ-1 (2.5 ng/ml), BMP4 (5 ng/ml), FGF2 (5 ng/ml), IGF-1 (50 ng/ml), Activin-A (10 ng/ml), Cardiotrophin (1 ng/ml), α-thrombin (1 U/ml), and Cardiogenol C (100 nM) in order to derive a cardiopoietic population.

The present invention allows the quantitative assessment of the cardio-generative potential of said cardiopoietic population, by quantifying the expression of two or more genes at the RNA level. This invention obviates the problems of qualitative observations, issue of time, and operator-dependence, inherent to the observation of subcellular location of transcription factor polypeptides. One method of choice is real-time quantitative reverse transcription polymerase chain reaction (qPCR). This method gives faster results (within one day) that are operator-independent and quantified relative to a reference standard. In addition, while immunostained samples require one-by-one fluorescent microscopy evaluation, up to 48 different samples (or conditions) can be tested in duplicate by qPCR using 96-well plates.

In order to identify suitable markers for qPCR, mRNA was extracted from cardiopoietic cells that were evaluated by immunofluorescence staining.

The reference standard consisted of cells from the same batch cultured in the absence of the cardiogenic cocktail.

Genes listed in Table 1, which are representative of cardiac transcriptional activity were evaluated.

qPCR was performed using a TaqMan PCR kit with an Applied Biosystems 7,900HT Sequence Detection System (Applied Biosystems, Foster City, Calif.). TaqMan Gene Expression reactions were incubated in a 96-well plate and run in triplicate. The threshold cycle (C_{T}) was defined as the fractional cycle number at which fluorescence passes a fixed threshold. TaqMan C_{T} values were converted into relative fold changes determined using the 2^{-ΔΔC}_{T} method, normalized to a housekeeping gene expression, i.e. GAPDH (P/N 435,2662-0506003).

Results for treated cells were normalized to results obtained for the corresponding reference standard.

A CARPI, which is a function of the quantification of the expression of two or more genes of said cells, was calculated as a linear average of the expression at the RNA level of Nkx2.5, Tbx-5, MEF2C, GATA-4, GATA-6, MESP-1 and FOG-1 using a calculation spreadsheet (Microsoft Excel 2007®, Microsoft Corporation). The following formula was used:

$$CARPI = \frac{1}{n} {\sum }_{i = 1}^{i = n} RNA {level}_{i}$$

where ‘i’ represents the selected gene and ‘n’ represents the total number of genes selected, with a minimum of 2. In this particular example, n=7.

The cardio-generative potential of hMSC-derived cardiopoietic cells was evaluated in nude, immunocompromised mice (Harlan, Indianapolis, Ind.). The protocol was approved by the competent Institutional Animal Care and Use Committee.

Myocardial infarction was performed. Following a blinded design, one month post-infarction a total of 600,000 total viable naive hMSC or 600,000 total viable hMSC-derived cardiopoietic cells, suspended in 12.5 μl of platelet lysate-free propagation medium, were injected under microscopic visualization in five epicardial sites on the anterior wall of the left ventricle (2.5 μl per injection site).

Left ventricular function and structure were serially followed by transthoracic echocardiography (Sequoia 512; Siemens, Malvern, Pa. and VisualSonics Inc, Toronto, Canada). Left ventricular ejection fraction (LVEF, %) was calculated as [(LVVd−LVVs)/LVVd]×100, where LVVd is left ventricular end-diastolic volume (ηl), and LVVs is left ventricular end-systolic volume (μl).

A change of LVEF (ΔEF) was calculated as the difference between LVEF measured one month after cell injection and LVEF measured prior to cell injection.

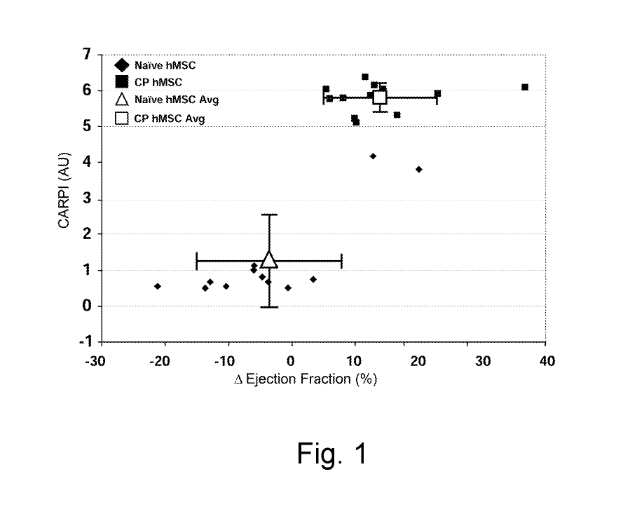

FIG. 1 is a graph plotting the CARPI for each individual cell culture against the corresponding ΔEF for the mouse injected with the respective said individual cell culture. Naive hMSC (small black diamonds) typically demonstrated a low CARPI associated with no significant improvement in myocardial function (negative ΔEF) one month post-cell injection. It is worth noting rare batches of naive hMSCs innately possessing high CARPI value together with an innate regenerative potential. The average for all batches of naive hMSCs is shown by a large white triangle. hMSC-derived cardiopoietic cells (small black squares) typically demonstrated an elevated CARPI associated with robust increase in myocardial function (positive ΔEF). The average for all batches of hMSC-derived cardiopoietic cells is shown by a large white square. Averages are represented together with the corresponding 95% confidence interval.

Thus, the inventors demonstrate that there is a positive correlation between an elevated CARPI of the cells to be injected and the change in ejection fraction after injection in the infarcted heart. Thus, the CARPI is a predictive index of cardio-generative potential.

TABLE 1

| Applied | | |
|---|---|---|
| Biosystems | | |
| Assay ID | Gene name | Gene symbol |
| Hs00231763_m1 | -Homeobox transcription factor or | Nkx2.5 or |
| | -NK2 transcription factor related, | NKX2-5 or |
| | locus 5 | NKX2.5 |
| Hs00171403_m1 | -Zinc finger cardiac transcription | GATA-4 or, |
| | factor or | GATA4 (AB) |
| | -GATA binding protein 4 | |
| Hs00231149_m1 | -Myocyte enhancer factor 2C | MEF2c or |
| | | MEF2C |
| Hs00361155_m1 | -T-box transcription factor or | Tbx5 or TBX5 |
| | -T-box 5 | |
| Hs00542350_m1 | -GATA co-factor | FOG 1 of |
| | (“Friend of GATA”) or | FOG-1 |
| | -zinc finger protein, multitype 1 | or FOG1 |
| Hs00251489_m1 | -Helix-loop-helix transcription | Mesp1 or |
| | factor | MESP1 |
| | -Mesoderm posterior 1 homolog | |
| | (mouse) (AB) | |
| Hs00232018_m1 | -GATA binding protein 6 (AB) | GATA-6 or |
| | | GATA6 |
| Hs00911699_m1 | -Kinase insert domain receptor (a | Flk-1, or |
| | type III receptor tyrosine kinase) | FLK1 or KDR |

### EXAMPLE 2

Similar results have been observed by treating stem cells with a cocktail containing recombinant TGFβ-1(2.5 ng/ml), BMP4 (5 ng/ml), Activin-A (5 ng/ml), FGF-2 (10 ng/ml), α-thrombin (1 U/ml), IGF-1 (50 ng/ml), Cardiotrophin (1 ng/ml) and Cardiogenol C (100 nM) used in a combinatorial fashion.

### EXAMPLE 3

Similar results have been observed by treating stem cells with a cocktail containing recombinant TGF-β1 (2.5 ng/ml), BMP-4 (5 ng/ml), Activin-A (5 ng/ml), FGF-2 (10 ng/ml), α-thrombin (1 U/ml), IGF-1 (50 ng/ml), IL-6 (100 ng/ml) and retinoic acid (1 μM) used in a combinatorial fashion.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. (canceled)

2. A method for preparing a composition comprising cardiopoietic cells, said method comprising the steps of:
a) obtaining stem cells;
b) treating the stem cells with a cardiogenic preparation to produce cardiopoietic cells;
c) determining the cardio-generative potential of the cardiopoietic cells, by:
i) collecting the expression level of two or more genes in the cardiopoietic cells, wherein said genes are selected from the group consisting of the following genes: Nkx2.5; Tbx5; MEF2C; GATA4; GATA6; Mesp1; FOG1; FOG2; Flk1; and homologues thereof;
ii) determining a CARdiac generation Potential Index (CARPI), the CARPI being a function of the quantification of the expression level of said two or more genes of said cells;
d) selecting cardiopoietic cells having an elevated CARPI compared to a reference CARPI of stem cells that have not been treated with the cardiogenic preparation; and
e) formulating the selected cardiopoietic cells into the composition.

3. The method of claim 2, wherein the stem cells are obtained from an individual suffering from a heart disease or disorder.

4. The method of claim 2, wherein said expression level of said two or more genes is quantified at the level of messenger RNAs (mRNAs), microRNAs, or a combination thereof.

5. The method of claim 2, wherein said expression level of said two or more genes is quantitatively measured at the level of messenger RNAs (mRNAs).

6. The method of claim 2, wherein said cardiopoietic cells contain no detectable sarcomeric proteins.

7. The method of claim 2, wherein said stem cells are mesenchymal stem cells.

8. The method of claim 2, wherein the CARPI is put into correlation with a parameter of cardiac function.

9. The method of claim 2, wherein the CARPI is measured to quantitatively assess the cardiogenic potential of a treatment.

10. A method for treatment comprising administering the composition of claim 2 to an individual in need thereof.
